(19) United States
(12) Patent Application Publication
(10) Pub. No.: US 2026/0041852 A1
(43) Pub. Date: Feb. 12, 2026

(54) NEEDLE SHIELD FOR SYRINGE

(71) Applicant: AMGEN INC., Thousand Oaks, CA (US)

(72) Inventors: Robert W. Swift, Fillmore, CA (US); James Nakamura, Montery Park, CA (US); Michael E. Akers, Thousand Oaks, CA (US); Jerome Olivas, Thousand Oaks, CA (US)

(21) Appl. No.: 19/100,412

(22) PCT Filed: Aug. 11, 2023
(86) PCT No.: PCT/US2023/030030

§ 371 Date: Jan. 31, 2025

Related U.S. Application Data
(60) Provisional application No. 63/399,131, filed on Aug. 18, 2022.

(51) Int. Cl. A61M 5/32

(52) U.S. Cl. CPC A61M 5/3202; A61M 5/3293; A61M 2205/0222

(57) ABSTRACT

A syringe assembly includes a syringe having a barrel and a needle hub disposed at a distal end of the syringe and a needle shield including an inner surface and a cavity for receiving the needle hub. The inner surface of the needle shield engaging an outer surface of the needle hub to form a sealing interface therebetween and removably couple the needle hub and the needle shield when the needle shield is disposed on the syringe. In some examples, the sealing interface comprises at least one contoured feature configured to limit or reduce an amount of force required to remove the needle shield from the syringe.

### CROSS-REFERENCE TO RELATED APPLICATIONS

Priority is claimed to U.S. Provisional Patent Application No. 63/199,131, filed Aug. 18, 2022, the entire contents of which are hereby incorporated by reference herein.

### FIELD OF THE DISCLOSURE

The present disclosure is generally directed to syringes, and more particularly, to needle shields for use in conjunction with syringes.

### BACKGROUND

A syringe may contain a drug and include a needle for delivering the drug into, for example, a patient. A needle shield may be provided to maintain sterility of the needle, protect the needle from damage, and/or reduce the likelihood of accidental needle stick injuries. To secure the needle shield to the syringe, the needle shield may be designed to frictionally couple with the syringe. Prior to use of the syringe for delivering the drug, the needle shield may need to be removed from the syringe. In at least some instances, the interface between the syringe and needle shield may create substantial resistance to a force applied to remove the needle shield from the syringe.

To remove the needle shield, a user may grab the needle shield and pull it off with their hands and/or employ a needle shield remover. Certain needle shield removers grab an end of the needle shield and the needle shield remover may provide a grip for the user.

### SUMMARY

Disclosed herein is a syringe assembly. The syringe assembly includes a syringe having a barrel and a needle hub disposed at a distal end of the syringe and a needle shield including an inner surface and a cavity for receiving the needle hub. The inner surface of the needle shield engaging an outer surface of the needle hub to form a sealing interface therebetween and removably couple the needle hub and the needle shield when the needle shield is disposed on the syringe. In some examples, the sealing interface comprises at least one contoured feature configured to limit or reduce an amount of force required to remove the needle shield from the syringe.

In some variations, at least one contoured feature is defined by the needle hub. In some such variations, the at least one contoured feature comprises at least one annular groove and/or at least one annular ridge defined in the needle hub surface. Alternatively, in other variations, at least one contoured feature comprises a plurality of dimples and/or a plurality of protrusions defined by the needle hub surface.

In other variations, at least one contoured surface is defined by the needle shield. For example, the at least one contoured feature may comprise at least one annular groove and/or at least one annular ridge defined by the needle shield surface. Alternatively, at least one contoured surface comprises a plurality of dimples and/or a plurality of protrusions defined by the needle shield surface.

Additionally disclosed herein is a reduced friction needle shield including a body having a proximal end and a distal end. The needle shield further includes an inner surface defining a cavity, and configured to contact an outer surface of a syringe to removably couple the needle shield and the syringe. The needle shield also includes an aperture formed in the proximal end of the body and communicating with the cavity. Additionally, the inner surface includes at least one contoured feature configured to limit or reduce an amount of force required to remove the needle shield from the syringe.

In some variations, the aperture of the cavity is a circular aperture, and the cavity defines a conical cavity portion and a cylindrical frictional portion disposed between the aperture and the conical cavity portion. Additionally, the needle shield surface and at least one contoured feature may be disposed in the cylindrical frictional portion of the cavity. For example, at least one contoured feature comprises at least one annular groove and/or at least one annular ridge.

Alternatively, at least one contoured feature comprises a plurality of dimples and/or a plurality of protrusions. In some examples, the plurality of dimples and/or a plurality of protrusions are uniformly distributed or non-uniformly distributed.

Also disclosed herein is a syringe, including a barrel having a proximal end and a distal end. The syringe further includes a needle hub disposed at the distal end of the barrel and comprising an outer surface configured to contact an inner surface of a needle shield to removably couple the needle shield and the syringe. Additionally, the outer surface of the needle hub comprises at least one contoured feature configured to limit or reduce a surface area of the outer surface of the needle hub in contact with the inner surface of the needle shield.

In some variations, the needle hub further comprises a syringe bulb disposed on a distal end of the needle hub. In some such variations, the syringe bulb defines a first radius and a needle hub neck defines a second radius, the first radius greater than the second radius.

In other variations, at least one contoured feature is disposed on the syringe bulb. For example, the at least one contoured feature may include an annular groove. Alternatively, the at least one contoured feature may include a plurality of dimples and/or a plurality of protrusions. In some such examples, the plurality of dimples and/or the plurality of protrusions are uniformly distributed or non-uniformly distributed.

Further disclosed herein is a reduced friction needle shield including an elongated body having a proximal end and a distal end, the elongated body constructed of a needle shield material. Additionally, the reduced friction needle shield includes an aperture defined in the proximal end of the needle shield. Further, the reduced friction needle shield may include a cavity disposed in the needle shield extending distally from the aperture, the cavity defined by a wall portion of the elongated body defining a needle shield surface for sealing against a needle hub of a syringe during use. The needle shield material may comprise a material having a hardness greater than approximately 50 Shore A.

In some variations, the aperture is a circular aperture and the cavity comprises a conical portion and a cylindrical portion disposed between the conical portion and the aperture, at least part of the cylindrical portion defining the needle shield surface. Additionally, in some examples, the needle shield surface maintains a substantially constant diameter under tensile loads applied to the elongated body in the range of approximately 0 N to approximately 10 N.

### BRIEF DESCRIPTION OF THE DRAWINGS

The present disclosure may be best understood by reference to the following description taken in conjunction with the accompanying drawings, in which:

FIG. 1 is a side view of a first example needle shield made in accordance with the present disclosure.

FIG. 2 is a side view of the first example needle shield of FIG. 1 coupled to a syringe.

FIG. 3 is a side view of a first example syringe made in accordance with the present disclosure.

FIG. 4 is a side view of the first example syringe of FIG. 3 coupled to a needle shield.

FIG. 5 is a side view of a second example syringe made in accordance with the present disclosure.

FIG. 6 is a side view of the second example syringe of FIG. 5 coupled to a needle shield.

FIG. 7 is a side view of a second example needle shield made in accordance with the present disclosure.

FIG. 8 is a side view of the second example needle shield of FIG. 7 coupled to a syringe.

FIG. 9 is a side view of a third example needle shield made in accordance with the present disclosure.

The figures depict preferred embodiments for purposes of illustration only and are not to scale. One skilled in the art will readily recognize from the following discussion that alternative embodiments of the systems and methods illustrated herein may be employed without departing from the principles described herein.

### DETAILED DESCRIPTION

Prefilled syringes filled with a drug and other syringes are often provided with a needle shield to maintain sterility of a needle of the syringe, protect this needle from damage, and/or reduce the likelihood of accidental needle stick injuries. The needle shield may be tightly secured to the syringe, which can present a challenge for at least some end users in remove the needle shield from the syringe. In some instances, removing a needle shield from a syringe can require a user to manually apply approximately 45 Newtons (N) or more of force, which is beyond the ability of some users.

A needle shield or syringe made in accordance with the present disclosure is configured to provide a limited or reduced friction interface between the needle shield and the syringe without compromising the intended objectives of providing the needle shield to, for example, maintain the sterility of a needle of the syringe, protect the needle from damage, and/or prevent accidental needle sticks. As a result, the reduced friction interface limits or reduces the amount of force required to remove the needle shield, thereby making it easier to remove the needle shield from the syringe as compared to conventional arrangements. As used herein, the term limit can mean setting a removal force upper threshold, for example, which is not to be exceeded. For example, in at least some embodiments, the force needed to remove the needle shield can be limited to less than or equal to approximately 5 N or less than or equal to approximately 10 N. The term “reduce” as used herein can mean minimizing the force required for needle shield removal while still serving the intended objectives and/or providing a removal force that is less than removal forces required for otherwise conventional arrangements. For example, in at least some embodiments, the needle shield or syringe of the present disclosure can reduce the friction force between the needle shield and the syringe when compared to conventional arrangements by reducing the surface area of contact between the syringe and the needle shield when compared to conventional arrangements.

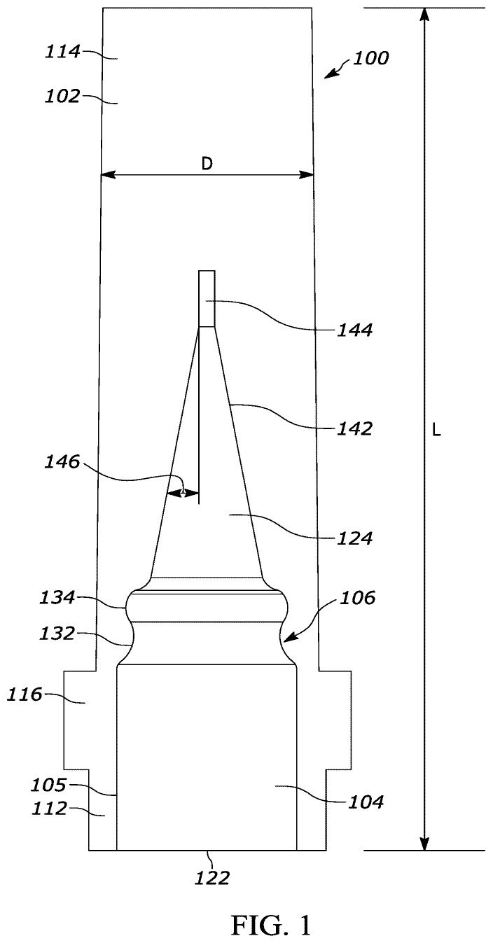

Turning to FIG. 1, the needle shield 100 is made in accordance with the present disclosure. As shown, the needle shield 100 includes a body 102, a cavity 104, and a contoured feature 106. As shown, the body 102 includes a diameter D and a length L, however, in various examples, the diameter D and length L can be larger or smaller than shown.

The body 102 is generally cylindrical and made of a thermoplastic material, but in other examples, the body 102 could be another shape or could be made of other natural or synthetic materials. As shown, the body 102 includes a proximal end 112 and a distal end 114. In the present example, the body 102 includes a shoulder 116. The shoulder 116 could be an annular shoulder 116 that wraps around the body proximate the proximal end 112. In other examples, the shoulder 116 may not wrap around the entire circumference of the body 102 or the shoulder 116 could be disposed elsewhere on the body, such as proximate the distal end 114.

The needle shield 100 further includes a cavity 104, defined by an inner surface 105 (or a needle shield surface) in the proximal end 112 and configured to receive a needle hub of a syringe. As shown, the cavity 104 includes an aperture 122, the contoured feature 106, and a conical cavity portion 124. In the present example, the aperture 122 is a circular aperture and opens into a cylindrical cavity portion 126. In other examples, the aperture 122 is an alternative cross-sectional shape. The contoured feature is disposed between the aperture 122 and the conical cavity portion 124.

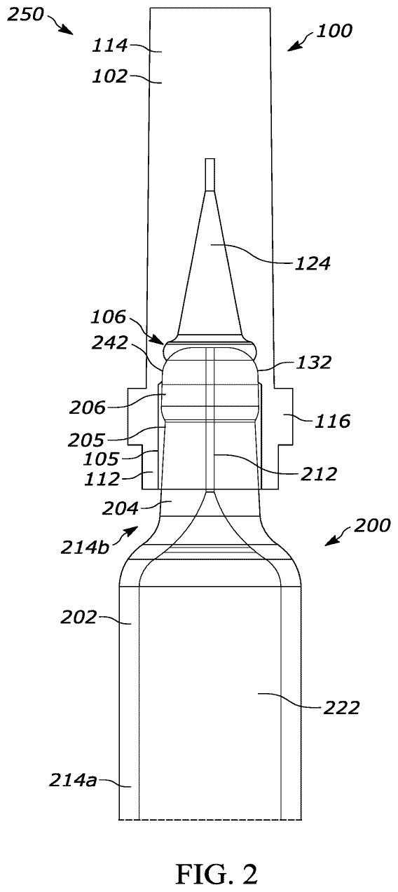

As illustrated in FIG. 1, the inner surface 105 is designed for engaging a needle hub outer surface 205 (discussed in greater detail in connection with FIG. 2). More specifically, the contoured feature 106 includes an annular ridge 132 defined by the inner surface 105. The annular ridge 132 is partially defined by an adjacent annular groove 134. As a result, the contoured feature 106 is designed to contact a syringe bulb along the annular ridge 132. The reduced surface area of contact is designed to reduce the friction force when removing the needle shield 100 from a syringe.

The needle shield 100 further includes the conical cavity portion 124. The conical cavity portion 124 includes an angled surface 142 that is configured to guide a needle into a needle receiving portion 144. The needle receiving portion 144 receives the needle and ensures sterility of the needle during syringe transportation and storage. The angled surface 142 of the conical cavity portion includes an angle 146 of approximately 10 degrees (°). However, in various other examples, the angle 146 could be greater or less than 10°, for example, the angle 146 could be as little as 2° or as high as 30°.

FIG. 2 illustrates a syringe assembly 250 including the needle shield 100 disposed on a syringe 200. In the present example, the syringe 200 includes a syringe barrel 202, a needle hub 204, and a syringe bulb 206 disposed on the needle hub 204. The syringe barrel 202 includes the container volume 222 and the needle hub 204 includes needle channel 212. As shown in FIG. 2, the needle hub 204 is disposed on a distal end 214b of the syringe 200, opposite the proximal end 214a. In various examples, the syringe 200 may include alternative structures or dimensions than shown in FIG. 2.

As shown in FIG. 2, the syringe 200 contacts the outer surface 205 of the needle shield 100 at the contoured feature 106. More particularly, the syringe bulb 206 of the needle hub 204 and the annular ridge 132 define a sealing interface 242. In accordance with the present disclosure, the sealing interface 242 provides sufficient friction to prevent the needle shield 100 from falling off the syringe 200 during transportation but not too much friction that an end user has difficulty removing the needle shield 100 from the syringe 200. To provide the desired amount of friction, the sealing interface 242 may constitute the only physical contact between the syringe 200 and the needle shield 100.

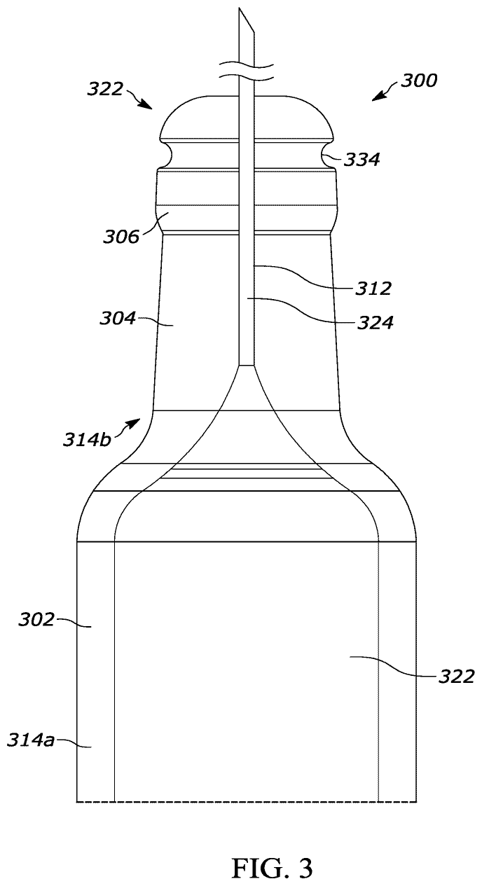

FIG. 3 illustrates a syringe 300 made in accordance with the present disclosure. The example syringe 300 includes a syringe barrel 302, a needle hub 304, and a syringe bulb 306. The syringe barrel 302 includes the container volume 322 and the needle hub 304 includes a needle 324 disposed in a needle channel 312. In other figures, the needle 324 is omitted for the sake of clarity. As shown in FIG. 3, the needle hub 304 is disposed on a distal end 314b of the syringe 300, opposite the proximal end 314a. In various examples, the syringe 300 may include alternative structures or dimensions than shown in FIG. 3.

The syringe bulb 306 includes a contoured feature 332. In the present example, the contoured feature 332 includes an annular groove 334. In the present example, the annular groove 334 circumscribes the syringe bulb 306.

However, in other examples, the annular groove 334 may comprise a plurality of grooves. Additionally, the annular groove 334 is shown to have a semi-circular cross section shape, but the annular groove 334 could have any cross-sectional shape, including semi-elliptical, rectangular, triangular, etc.

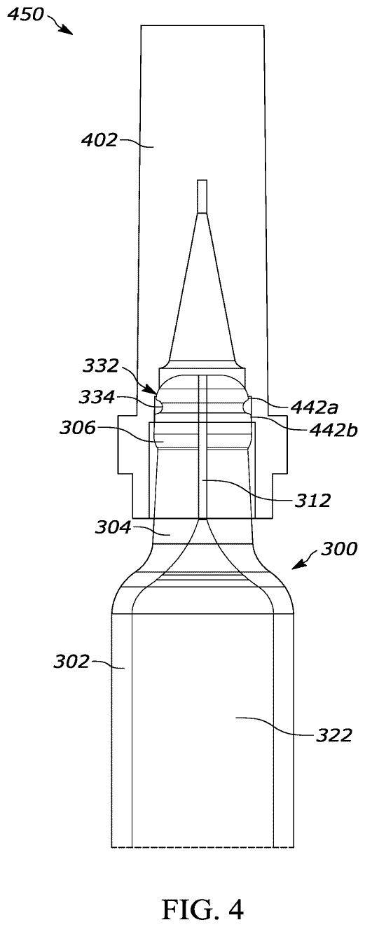

In FIG. 4, the syringe 300 is mechanically coupled to the needle shield 402, and forming a syringe assembly 450. The syringe is frictionally coupled to the needle shield 402 at the syringe bulb 306. Specifically, the sealing interface 442a, 442b is on either side of the annular groove 334. Therefore, the annular groove 334 reduces the surface area of contact between the syringe 300 and the needle shield 402. By reducing the surface area of contact between the syringe 300 and the needle shield 402, there is reduced friction between the needle shield 402 and the syringe bulb 306.

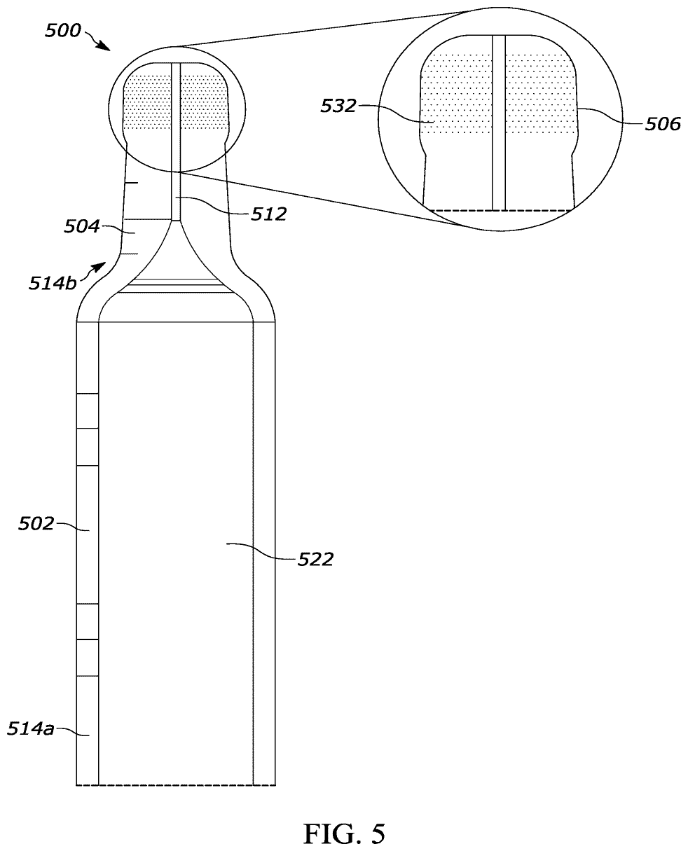

FIG. 5 illustrates an alternative embodiment of the present disclosure in which a syringe 500 includes an alternative configuration. The syringe 500 includes a syringe barrel 502, a needle hub 504, and a syringe bulb 506. As shown, the syringe barrel 502 includes a container volume 522 and a needle channel 512. As shown in FIG. 5, the needle hub 504 is disposed on a distal end 514b of the syringe 500, opposite the proximal end 514a. In various alternative examples, the syringe 500 can have alternative dimensions or configurations.

The syringe bulb 506 of the present embodiment includes a contoured surface 532 including a plurality of dimples and/or protrusions. In the illustrated example, the contoured surface 532 covers substantially the entire surface of the syringe bulb 506. In other examples, the contoured surface 532 may cover less of the syringe bulb 506 (e.g., cover approximately 50% of the surface, 25% of the surface, etc.). Further, the dimple size and distribution could be greater or lesser than shown in FIG. 5. For example, the contoured surface 532 may include more or fewer dimples per square inch. Additionally, the dimples may be larger or smaller than shown.

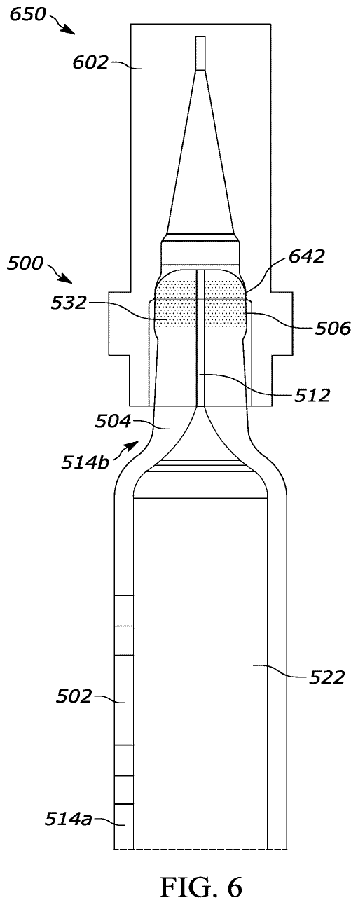

FIG. 6 illustrates a syringe assembly 650 including the syringe 500 mechanically coupled to a needle shield 602. the syringe 500 is mechanically coupled to the needle shield 602. The syringe 500 is frictionally coupled to the needle shield 602 at the syringe bulb 506. Specifically, the sealing interface 642 around the contoured surface 532. The contoured surface 532 reduces the surface area of contact between the syringe 500 and the needle shield 602. By reducing the surface area of contact between the syringe 500 and the needle shield 602, there is reduced friction between the needle shield 602 and the syringe bulb 506.

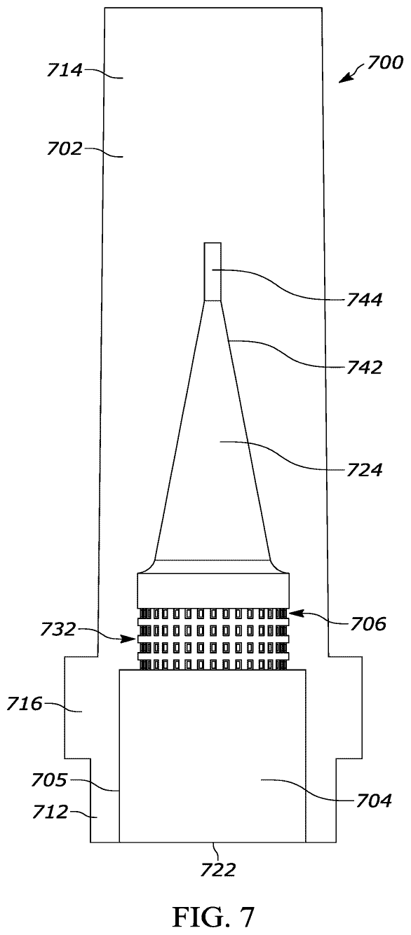

FIG. 7 illustrates a needle shield 700 made in accordance with the present disclosure. The needle shield 700 includes a body 702, a cavity 704, and a contoured surface 706.

The body 702 is generally cylindrical and made of a thermoplastic material, but in other examples, the body 702 could be another shape or could be made of other natural or synthetic materials. As shown, the body 702 includes a proximal end 712 and a distal end 714. In the present example, the body 702 includes a shoulder 716. The shoulder 716 could be an annular shoulder 716 that wraps around the body proximate the proximal end 712. in other examples, the shoulder 716 may not wrap around the entire circumference of the body 702 or the shoulder 716 could be disposed elsewhere on the body, such as proximate the distal end 714.

The needle shield 700 further includes a cavity 704, defined by an inner surface 705, configured to receive a syringe. As shown, the cavity 704 includes an aperture 722, the contoured feature 706, and a conical cavity portion 724. In the present example, the aperture 722 opens into a cylindrical cavity portion 726. The contoured feature is disposed between the aperture 722 and the conical cavity portion 724.

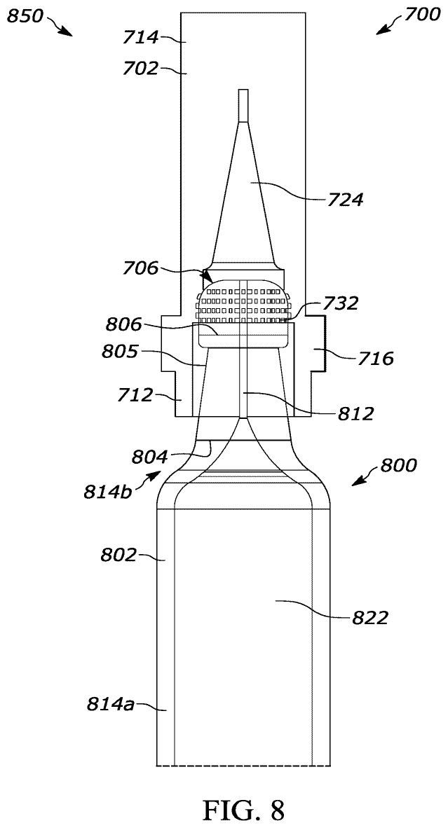

As illustrated in FIG. 7, the inner surface 705 is designed for engaging a needle hub outer surface 805 (discussed in greater detail in connection with FIG. 8). More specifically, the contoured feature 706 includes a protrusion array 732. The protrusion array 732 includes a plurality of rigid or semi-rigid protrusions 734. In some examples, the protrusion array 732 could, alternatively, be made of a plurality of dimples. The contoured feature 706 is configured to contact a syringe bulb at each of the protrusions 734, thereby reducing the surface area of contact between the needle shield 700 and a syringe.

The needle shield 700 further includes the conical cavity portion 724. The conical cavity portion 724 includes an angled surface 742 that is configured to guide a needle into a needle receiving portion 744. The needle receiving portion 744 receives the needle and ensures sterility of the needle during syringe transportation and storage. The angled surface 742 of the conical cavity portion includes an angle 746 of approximately 10 degrees (°). However, in various other examples, the angle 746 could be greater or less than 10°, for example, the angle 746 could be as little as 2° or as high as 30°.

As shown in FIG. 8, the needle shield 700 is disposed on syringe 800, thereby forming a syringe assembly 850. In the present example, the syringe 800 includes a syringe barrel 802, a needle hub 804, and a syringe bulb 806. The syringe barrel 802 includes the container volume 822 and the needle hub 804 includes needle channel 812. As shown in FIG. 8, the needle hub 804, including an outer surface 805, is disposed on a distal end 814b of the syringe 800, opposite the proximal end 814a. In various examples, the syringe 800 may include alternative structures or dimensions than shown in FIG. 2.

As shown in FIG. 8, the syringe 800 contacts the outer surface 805 of the needle shield 800 at the contoured feature 706. More particularly, the syringe bulb 806 of the needle hub 804 and the protrusion array 732 define a sealing interface 842. In accordance with the present disclosure, the sealing interface 842 provides sufficient friction to prevent the needle shield 700 from falling off the syringe 800 during transportation but not too much friction that an end user has difficulty removing the needle shield 700 from the syringe 800. To provide the desired amount of friction, the sealing interface 842 may constitute the only physical contact between the syringe 800 and the needle shield 700.

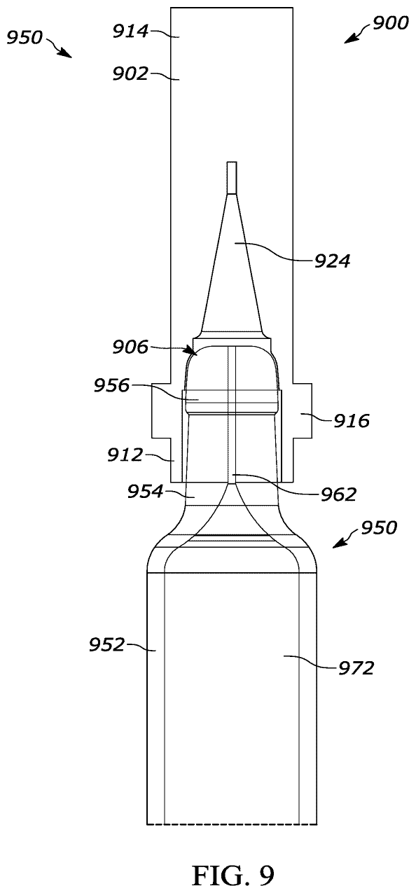

FIG. 9 illustrates yet another alternative syringe assembly 950 including needle shield 900 made in accordance with the present disclosure. The alternative needle shield 900 is made of a harder material than typical needle shields. In some examples, the needle shield 900 is made of a harder material than the needle shields 100, 400, 600, and 700 of the present disclosure. As shown, the needle shield 100 includes a body 102, a cavity 104, and a contoured feature 106.

The body 902 is generally cylindrical and made of a thermoplastic material, but in other examples, the body 902 could be another shape or could be made of other natural or synthetic materials. As shown, the body 902 includes a proximal end 912 and a distal end 914. In the present example, the body 902 includes a shoulder 916. The shoulder 916 could be an annular shoulder 916 that wraps around the body proximate the proximal end 912. in other examples, the shoulder 916 may not wrap around the entire circumference of the body 902 or the shoulder 916 could be disposed elsewhere on the body, such as proximate the distal end 914.

The needle shield 900 further includes a cavity 904 configured to receive a syringe. As shown, the cavity 904 includes an aperture 922 and a conical cavity portion 924. In the present example, the aperture 922 opens into a cylindrical cavity portion 926. In other examples, the aperture 122 is an alternative cross-sectional shape. In the present example, the needle shield 900 reduces friction between the needle shield 900 and syringe 950 because the needle shield 900 is made of a more rigid material that has reduced deformation under axial loads. As a result, the aperture 922 does not deform and increase friction on the syringe 950. In some such examples, the needle shield 900 is made of a material having a hardness greater than approximately 50 Shore A. With such a material, the cavity 904 and needle shield surface 905 maintain a substantially constant diameter under tensile loads in the range of approximately 0 N to approximately 20 N. As used herein, the diameter substantially deforms if the diameter is reduced more than 5 percent from an undeformed state.

The needle shield 900 further includes the conical cavity portion 924. The conical cavity portion 924 includes an angled surface 942 that is configured to guide a needle into a needle receiving portion 944. The needle receiving portion 944 receives the needle and ensures sterility of the needle during syringe transportation and storage. The angled surface 942 of the conical cavity portion includes an angle 946 of approximately 10 degrees (°). However, in various other examples, the angle 946 could be greater or less than 10°, for example, the angle 946 could be as little as 2° or as high as 30°.

In each of the foregoing embodiments, the needle shield and syringe needle hub are manufactured to reduce the friction that must be overcome to remove the needle shield from the syringe. The friction is reduced because the surface area of contact between the needle shield and the syringe is reduced. In at least some embodiments, the friction may be reduced to a level where the force needed to remove the needle shield from the syringe may be less than or equal to approximately 5 Nor less than or equal 10 N. Additionally, the friction reducing feature(s) described herein may be added one of or both of the needle shield and the syringe and/or combined to further improve the friction reducing effects in accordance with the present disclosure.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like;Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5p1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGp mAb (MDX-1307); anti-mesothelin dsFv-PE38 conjugate (CAT-5001); anti-PD1mAb (MDX-1106 (ONO-4538)); anti-PDGFRa antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1 BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vll (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A syringe assembly, comprising:
a syringe comprising a barrel and a needle hub disposed at a distal end of the syringe;
a needle shield comprising an inner surface and a cavity for receiving the needle hub, the inner surface of the needle shield engaging an outer surface of the needle hub to form a sealing interface therebetween and removably couple the needle hub and the needle shield when the needle shield is disposed on the syringe; and
wherein the sealing interface comprises at least one contoured feature configured to limit or reduce an amount of force required to remove the needle shield from the syringe.

2. The syringe assembly of claim 1, wherein the at least one contoured feature is defined by the needle hub.

3. The syringe assembly of claim 1, wherein the at least one contoured feature comprises at least one annular groove and/or at least one annular ridge defined by the outer surface of the needle hub.

4. The syringe assembly of claim 1, wherein the at least one contoured feature comprises a plurality of dimples and/or a plurality of protrusions defined by the outer surface of the needle hub.

5. The syringe assembly of claim 1, wherein the at least one contoured feature is defined by the needle shield.

6. The syringe assembly of claim 1, wherein the at least one contoured feature comprises at least one annular groove and/or at least one annular ridge defined by the inner surface of the needle shield.

7. The syringe assembly of claim 1, wherein the at least one contoured feature comprises a plurality of dimples and/or a plurality of protrusions defined by the inner surface of the needle shield.

8. A needle shield, comprising:
a body including a proximal end and a distal end;
an inner surface defining a cavity and configured to contact an outer surface of a syringe to removably couple the needle shield and the syringe;
an aperture formed in the proximal end of the body and communicating with the cavity; and
wherein either (a) or (b):
(the inner surface comprises at least one contoured feature configured to limit or reduce an amount of force required to remove the needle shield from the syringe, or
(b) the needle shield material comprises a material having a hardness greater than approximately 50 Shore A.

9. The needle shield of claim 8, wherein the aperture is a circular aperture, and the cavity comprises a conical cavity portion and a cylindrical portion disposed between the aperture and the conical cavity portion.

10. The needle shield of claim 9, comprising (a) and wherein the inner surface of the needle shield and the at least one contoured feature are disposed in the cylindrical portion of the cavity.

11. The needle shield of claim 8, comprising (a) and wherein the at least one contoured feature comprises at least one annular groove and/or at least one annular ridge.

12. The needle shield of claim 8, comprising (a) and wherein the at least one contoured feature comprises a plurality of dimples and/or a plurality of protrusions

13. The needle shield of claim 12, wherein the plurality of dimples and/or a plurality of protrusions are uniformly distributed or non-uniformly distributed.

14. A syringe, comprising:
a barrel including a proximal end and a distal end;
a needle hub disposed at the distal end of the barrel and comprising an outer surface configured to contact an inner surface of a needle shield to removably couple the needle shield and the syringe; and
wherein the outer surface of the needle hub comprises at least one contoured feature configured to limit or reduce a surface area of the outer surface of the needle hub in contact with the inner surface of the needle shield.

15. The syringe of claim 14, wherein a distal end of the needle hub comprises a syringe bulb, the syringe bulb defining a first radius and a needle hub neck defining a second radius, the first radius greater than the second radius.

16. The syringe of claim 15, wherein the syringe bulb comprises the at least one contoured feature.

17. The syringe of claim 15, wherein the at least one contoured feature comprises an annular groove.

18. The syringe of claim 15, wherein the at least one contoured feature comprises a plurality of dimples and/or a plurality of protrusions.

19. The syringe of claim 18, wherein the plurality of dimples and/or the plurality of protrusions are uniformly distributed or non-uniformly distributed.

20. (canceled)

21. (canceled)

22. The needle shield of claim 20, comprising (b) and wherein the inner surface of the needle shield maintains at least a substantially constant diameter under tensile loads applied to the body in the range of approximately 0 N to approximately 10 N.
